(19) United States
(12) Patent Application Publication
(10) Pub. No.: US 2026/0091122 A1
(43) Pub. Date: Apr. 2, 2026

(54) Hybrid Molecule Comprising an Antibody FC Portion and at Least One Peptide Binding to a Self-Reactive Lymphocyte Involved in Autoimmune Dermatitis, and Uses Thereof

(71) Applicants: UNIVERSITE TOULOUSE III – PAUL SABATIER, Toulouse (FR); INSTITUT NATIONAL DE LA SANTÉ ET DE LA RECHERCHE MÉDICALE, Paris (FR); CENTRE HOSPITALIER UNIVERSITAIRE DE TOULOUSE, Toulouse (FR); CENTRE HOSPITALIER UNIVERSITAIRE DE MONTPELLIER, Montpellier (FR); UNIVERSITE DE MONTPELLIER, Montpellier (FR); ARTHRITIS RECHERCHE & DEVELOPPEMENT, Neuilly sur Seine (FR)

(72) Inventors: Guy Serre, Toulouse (FR); Cyril Clavel, Saint Foy de Peyrolieres (FR); Caroline Carle, Toulouse (FR)

(73) Assignees: UNIVERSITE TOULOUSE III – PAUL SABATIER, Toulouse (FR); INSTITUT NATIONAL DE LA SANTÉ ET DE LA RECHERCHE MÉDICALE, Paris (FR); CENTRE HOSPITALIER UNIVERSITAIRE DE TOULOUSE, Toulouse (FR); CENTRE HOSPITALIER UNIVERSITAIRE DE MONTPELLIER, Montpellier (FR); UNIVERSITE DE MONTPELLIER, Montpellier (FR); ARTHRITIS RECHERCHE & DEVELOPPEMENT, Neuilly sur Seine (FR)

(21) Appl. No.: 19/111,946

(22) PCT Filed: Sep. 15, 2023
(86) PCT No.: PCT/FR2023/051403

§ 371 Date: Mar. 14, 2025

(30) Foreign Application Priority Data
Sep. 15, 2022 (FR) FR2209320

(51) Int. Cl. A61K 47/68; A61P 37/04; C07K 14/78

(52) U.S. Cl. CPC A61K 47/6811; A61K 47/68; A61K 47/6889; A61P 37/04; C07K 14/78

(57) ABSTRACT

The invention relates to a hybrid molecule comprising at least one antibody Fc fragment covalently bonded to at least one peptide bonding to an autoreactive lymphocyte, responsible for autoimmune dermatitis, the uses of such a hybrid molecule, as well as the production method thereof.

The present invention relates to a hybrid molecule comprising at least one antibody Fc fragment covalently bonded to at least one peptide bonding to an autoreactive lymphocyte, involved in autoimmune dermatitis, the uses of such a hybrid molecule, as well as the production method thereof. Autoreactive lymphocytes express receptors on their surface. Said autoreactive lymphocyte receptors are membrane receptors of a B-lymphocyte (BCR) or a T-lymphocyte (TCR).

### BACKGROUND OF THE INVENTION

Autoimmune dermatitis is a disease of the skin and mucous membranes, in particular bonded to the production of autoantibodies by the body and/or pro-inflammatory cytokines secreted by T-lymphocytes.

Autoimmune dermatitis is often classified based on the involved autoantibody. For example, the presence of class G autoantibodies directed against desmogleins (the proteins of the interkeratinocyte desmosomes) is highly specific for pemphigus. On the contrary, the presence of anticollagen XVII autoantibodies is rather representative of bullous pemphigoid.

These autoantibodies are at the heart of autoimmune reactions specific to autoimmune dermatitis (eg Di Zenzo G, Thoma-Uszynski S, Calabresi V, Fontao L, Hofmann S C, Lacour J P, Sera F, Bruckner-Tuderman L, Zambruno G, Borradori L, Hertl M. Demonstration of epitope-spreading phenomena in bullous pemphigoid: results of a prospective multicenter study. J Invest Dermatol. 2011 November; 131(11): 2271-80. doi: 10.1038/jid.2011.180. Epub 2011 Jun. 23. PMID: 21697892; or Peng B, Temple B R, Yang J, Geng S, Culton D A, Qian Y. Identification of a primary antigenic target of epitope spreading in endemic pemphigus foliaceus. J Autoimmun. 2021 January; 116:102561. doi: 10.1016/j.jaut.2020.102561. Epub 2020 Nov. 4. PMID: 33158670; PMCID: PMC7770069). These autoantibodies are therefore a key therapeutic target.

The antigenic targets of the autoantibodies responsible for autoimmune dermatitis have been profiled and can be found in the IEDB database, Immune Epitope DataBase and analysis resource, http://www.iedb.org/home_v3.php. Said autoantibodies are in particular specifically directed against the α-1 polypeptide chain of collagen XVII, the polypeptide sequence of desmoglein-1, or even the polypeptide sequence of desmoglein-3.

To this day, there is no specific treatment for autoimmune dermatitis. Treatments usually involve administering systemic corticosteroids or even immunosuppressive drugs, which can cause serious side effects. Anti-CD20 antibodies may also be used, but these are not specific for the B-cells behind autoreactive antibodies and have no effect on autoreactive T-cells.

An object of the present invention is thus to provide a more targeted treatment of autoimmune dermatitis. By targeting B-cells behind autoreactive antibodies (autoantibodies) specific to autoimmune dermatitis, and/or autoreactive T-lymphocytes sources of pro-inflammatory cytokines, the present invention thus aims to provide a suitable treatment for each autoimmune dermatitis.

The present invention is based on the Inventors' research showing that it is possible to target the autoreactive lymphocyte receptors that recognize self peptides involved in the development of autoimmune dermatitis. More particularly, the present invention is based on the Inventors' research showing that it is possible to target the B-lymphocyte clones expressing the autoreactive lymphocyte receptors involved in autoimmune dermatitis and/or autoreactive T-lymphocytes (by bonding a self peptide involved in the development of autoimmune dermatitis to the TCR), and to eliminate them using a hybrid molecule comprising (i) at least one peptide bonding to an autoreactive lymphocyte involved in autoimmune dermatitis and (ii) a human immunoglobulin Fc fragment. These hybrid molecules will specifically target B-lymphocyte clones expressing autoantibodies responsible for autoimmune dermatitis and/or autoreactive T-lymphocytes (using said peptide, which is recognized by the B- and/or T-membrane receptors expressed by said B- and/or T-lymphocytes) which will then be eliminated, after binding the Fc fragment to the Fc receptors, by macrophages (via phagocytosis) and/or NK-cells (via antibody-dependent cell-mediated cytotoxicity—ADCC), and/or by complement cascade activation.

By targeting B-lymphocyte clones expressing autoantibodies responsible for autoimmune dermatitis and cells that differentiate into plasma cells that themselves secrete said autoantibodies and/or autoreactive T-lymphocytes sources of pro-inflammatory cytokines, the hybrid molecules of the invention thus aim to eliminate these “pathogenic” autoantibodies and the source of undesirable inflammation from the patients' bodies.

### DISCLOSURE OF THE INVENTION

### Hybrid Molecule According to the Invention

In a first aspect, the invention relates to a hybrid molecule comprising at least one antibody Fc fragment covalently bonded to at least one peptide bonding to an autoreactive lymphocyte involved in autoimmune dermatitis, at least one spacer being optionally present between said Fc fragment and said peptide. The outline of such a structure is shown in FIG. 1.

According to the invention, a “hybrid molecule” means a molecule having at least two components of different nature, namely the antibody Fc fragment and said peptide.

According to the invention, an “Fc fragment” of an antibody means the constant region of an immunoglobulin excluding the first immunoglobulin constant region domain (i.e. CH1-CL). Thus, the Fc fragment refers to a homodimer, each monomer comprising the last two constant domains of IgA, IgD, IgG (i.e. CH2 and CH3), or the last three constant domains of IgE and IgM (i.e., CH2, CH3 and CH4).

According to the invention, the expression “covalently bonded” means a covalent bond, that is to say a chemical bond in which two atoms share two electrons. Said covalent bond can be polar or non-polar.

According to the invention, a “spacer” is a bonding agent which makes it possible to covalently bond an Fc fragment of an antibody to said peptide bonding to an autoreactive lymphocyte involved in autoimmune dermatitis, while distancing said Fc fragment from said peptide (thereby reducing possible steric hindrance). It can be any molecule, and in particular a peptide or a polypeptide. Preferably, the spacer does not modify the physicochemical properties of the hybrid molecule.

The presence of at least one spacer is advantageous: it facilitates the independent accessibility of the two partners of the hybrid molecule (the Fc fragment is more easily accessible to bond with the Fc receptors, just as said peptide is more easily accessible to bond with the autoreactive lymphocytes), and/or stabilizes the hybrid molecule, and/or increases the solubility of the hybrid molecule.

According to an embodiment, the hybrid molecule according to the invention may comprise one or more spacers. Preferably, the hybrid molecule comprises one or two spacers. According to an embodiment, when at least one spacer is present in said hybrid molecule of the invention, said Fc fragment is covalently bonded to a spacer, said spacer itself being covalently bonded to said peptide. According to another embodiment, when at least two spacers are present in said hybrid molecule of the invention, said Fc fragment is covalently bonded to a first spacer, said first spacer itself being covalently bonded to a second spacer and the second spacer itself is covalently bonded to said peptide. The bond between the Fc fragment and the peptide can therefore be direct, or indirect in the presence of spacers.

According to an embodiment, the hybrid molecule according to the invention can comprise at least one peptide bonding to an autoreactive lymphocyte involved in autoimmune dermatitis. This means that the Fc fragment can be bonded to one or two peptides. Indeed, the Fc fragment comprises two monomers, and the Fc fragment can thus be covalently bonded to a peptide on only one of the two monomers, or the Fc fragment can be covalently bonded to a peptide on each monomer. Preferably, when two peptides are bonded with the Fc fragment, the two peptides are identical.

According to an embodiment, said spacer is a polymer containing one or more repeating units containing the ether group. According to a particular embodiment, said spacer is polyethylene glycol of formula PEG-n, wherein n denotes an integer between 1 and 100, preferably between 1 and 10, and in particular 1, 2, 3, 4 or 8. According to the invention, said polyethylene glycol may be functionalized, for example with an amine group (PEG-n-amine such as PEG-NH₂). According to the invention “an integer between 1 and 100” encompasses all integer values between 1 and 100, i.e.; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100.

The expression “autoreactive lymphocyte receptor involved in autoimmune dermatitis” means a receptor on the surface of a T-lymphocyte and/or a B-lymphocyte that recognizes a self peptide involving the activation of cells leading to autoimmune dermatitis by inappropriate production of autoantibodies or pro-inflammatory cytokines. More particularly, said lymphocyte receptor is a B-lymphocyte receptor (BCR) and/or a T-lymphocyte receptor (TCR). The bond of the peptide to the BCR and/or TCR is thus involved in the development of autoimmune dermatitis. For example, in the case of bullous pemphigoid the involved self peptide may comprise all or part of collagen XVII, and the involved autoantibody is an autoantibody directed against collagen XVII. Typically, binding anticollagen XVII autoantibodies disrupts the cohesion between the epidermis and the dermis, which causes a blister (“and consequently bullous pemphigoid”) to form.

The term “autoreactive lymphocyte involved in autoimmune dermatitis” means a lymphocyte expressing on its surface a receptor that recognizes a self peptide involving the activation of cells leading to autoimmune dermatitis by inappropriate production of autoantibodies or pro-inflammatory cytokines. Preferably, it is a T-lymphocyte and/or a B-lymphocyte.

In an embodiment, the term “peptide bonding to an autoreactive lymphocyte involved in autoimmune dermatitis” means a peptide comprising all or part of the polypeptide sequence of collagen XVII, all or part of the polypeptide sequence of desmoglein-1 (DSG1) and/or all or part of the polypeptide sequence of desmoglein-3 (DSG3). In an embodiment, the term “peptide bonding to an autoreactive lymphocyte involved in autoimmune dermatitis” means a peptide comprising all or part of the polypeptide sequence of collagen XVII, all or part of the polypeptide sequence of desmoglein-1 (DSG1) or all or part of the polypeptide sequence of desmoglein-3 (DSG3). Preferably, it is a peptide comprising all or part of the α-1 polypeptide chain of collagen XVII, the polypeptide sequence of desmoglein-1 (DSG1) or the polypeptide sequence of desmoglein-3 (DSG3).

According to an embodiment, the peptide according to the invention comprises all or part of the α-1 polypeptide chain of collagen XVII. Preferably, said α-1 polypeptide chain of collagen XVII is represented by SEQ ID NO: 35.

According to another embodiment, the peptide according to the invention comprises all or part of the polypeptide sequence of desmoglein-1 (DSG1). Preferably, said polypeptide sequence of DSG1 is represented by SEQ ID NO: 36.

According to another embodiment, the peptide according to the invention comprises all or part of the polypeptide sequence of desmoglein-3 (DSG3). Preferably, said polypeptide sequence of DSG3 is represented by SEQ ID NO: 37.

In an embodiment, a “peptide bonding to an autoreactive lymphocyte involved in autoimmune dermatitis” means a peptide recognized by an autoantibody directed against collagen XVII, a peptide recognized by an autoantibody directed against DSG1 or a peptide recognized by an autoantibody directed against DSG3. In an embodiment, a “peptide bonding to an autoreactive lymphocyte involved in autoimmune dermatitis” means a peptide recognized by an autoantibody directed against the α-1 chain of collagen XVII, a peptide recognized by an autoantibody directed against DSG1 or a peptide recognized by an autoantibody directed against DSG3. Such peptides can be obtained from fragments of collagen XVII (preferably the α-1 chain), DSG1, or DSG3, whether these fragments are natural, recombinant or synthetic. Such peptides can also be directly synthesized. The amino acids constituting the peptide may be of the L or D series, preferably of the L series. A peptide according to the invention bonds to an autoantibody directed against type XVII collagen (preferably the α-1 chain), against DSG1 or against DSG3, and the bonding between said peptide and the autoantibody can for example be verified using an ELISA test. (see for example “Dermatoses bulleuses auto-immunes: les auto-anticorps impliqués”, i.e., “Autoimmune bullous dermatoses: the involved autoantibodies”, Dr AS. Deleplancque Pr S. Dubucquoi, https://biologiepathologie.chu-lille.fr/fichiers/377_Dermatoses%20bulleuses%20nov%202017.pdf).

According to an embodiment, in said hybrid molecule according to the invention, said peptide comprises all or part of the polypeptide sequence of desmoglein 1, of the polypeptide sequence of desmoglein 3, or of the α-1 chain of type XVII collagen. Even more particularly, said desmoglein 1, said desmoglein 3, or said α-1 chain of type XVII collagen is of mammalian origin and 20 is preferably of human origin.

According to an embodiment, in said hybrid molecule according to the invention, the peptide has a size of at least 2 consecutive amino acids, 3 consecutive amino acids, 4 consecutive amino acids, preferably at least 5 consecutive amino acids. According to an embodiment, said peptide has a size of between 5 and 70 amino acids, in particular between 10 and 70. According to the invention, “between 5 and 70” means all values: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70.

According to an embodiment, in said hybrid molecule according to the invention, the peptide is linear.

According to an embodiment, in said hybrid molecule according to the invention, the peptide can be modified so as to improve its reactivity towards autoreactive lymphocytes. For example, peptides can be cyclized, peptides can be of the retro type (the L series amino acids are chained in a sequence that is the reverse of that of the peptide to replicate), or of the retro-inverso type (the amino acids are of the D type instead of the natural L series and are chained in a sequence that is the reverse of that of the peptide to Dubuc quoi replicate). According to an even more particular embodiment, in said hybrid molecule according to the invention, the terminal carboxyl function (COOH) of said peptide is replaced by a carboxamide function (CONH₂).

According to another embodiment, in said hybrid molecule according to the invention, the peptide can be modified so as to facilitate its synthesis and/or improve its stability, for example by alkylation. According to an even more particular embodiment, in said hybrid molecule according to the invention, the terminal amine function (NH₂) of said peptide is acetylated.

According to an embodiment, in said hybrid molecule according to the invention, the amine and carboxyl functions of the peptide can be in the form of the salt corresponding to the acid or to the base.

According to an even more particular embodiment, in said hybrid molecule according to the invention, said peptide is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.

According to an even more particular embodiment, in said hybrid molecule according to the invention, said peptide is selected from the group consisting of: SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 et SEQ ID NO: 48.

According to an even more particular embodiment, in said hybrid molecule according to the invention, said peptide is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 et SEQ ID NO: 48.

According to an even more particular embodiment, in said hybrid molecule according to the invention, said peptide is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 (Collagen XVII).

According to an even more particular embodiment, in said hybrid molecule according to the invention, said peptide is selected from the group consisting of: SEQ ID NO: 38, SEQ ID NO: 39, SEQ ID NO: 40, preferably SEQ ID NO: 38 or SEQ ID NO: 39.

According to an even more particular embodiment, in said hybrid molecule according to the invention, said peptide is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 38, SEQ ID NO: 39 and SEQ ID NO: 40 (Collagen XVII).

According to an even more particular embodiment, in said hybrid molecule according to the invention, said peptide is selected from the group consisting of: SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21 (DSG1).

According to an even more particular embodiment, in said hybrid molecule according to the invention, said peptide is selected from the group consisting of: SEQ ID NO: 41, SEQ ID NO: 42, SEQ ID NO: 43 and SEQ ID NO: 44 (DSG1).

According to an even more particular embodiment, in said hybrid molecule according to the invention, said peptide is selected from the group consisting of: SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 41, SEQ ID NO: 42, SEQ ID NO: 43 and SEQ ID NO: 44 (DSG1).

According to an even more particular embodiment, in said hybrid molecule according to the invention, said peptide is selected from the group consisting of: SEQ ID NO: 10, SEQ ID NO: 41, SEQ ID NO: 42, SEQ ID NO: 43 and SEQ ID NO: 44 (DSG1).

According to an even more particular embodiment, in said hybrid molecule according to the invention, said peptide is selected from the group consisting of: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30 (DSG3).

According to an even more particular embodiment, in said hybrid molecule according to the invention, said peptide is selected from the group consisting of: SEQ ID NO: 45, SEQ ID NO: 46, SEQ ID NO: 47 et SEQ ID NO: 48 (DSG3).

According to an even more particular embodiment, in said hybrid molecule according to the invention, said peptide is selected from the group consisting of: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 45, SEQ ID NO: 46, SEQ ID NO: 47 et SEQ ID NO: 48 (DSG3).

According to an even more particular embodiment, in said hybrid molecule according to the invention, said peptide is selected from the group consisting of: SEQ ID NO: 26, SEQ ID NO: 45, SEQ ID NO: 46, SEQ ID NO: 47 et SEQ ID NO: 48 (DSG3).

According to an embodiment, in said hybrid molecule according to the invention, said Fc fragment is a human Fc fragment, in particular of IgG, more particularly of IgG1. IgG1 can correspond to any allotypic variant, for example G1m3 or G1m17. For example, the Fc fragment of IgG1 is represented by SEQ ID NO: 31, SEQ ID NO: 32 (Fc+Qtag) or SEQ ID NO: 33 (Fc+Qtag bis).

According to an embodiment, in said hybrid molecule according to the invention, said Fc fragment is a wild-type or mutated fragment. The mutation(s) may aim to increase the plasma half-life, decrease it, or modify the effector functions of the Fc fragment. According to an even more particular embodiment, said mutated Fc fragment comprises at least the following mutations:
L234A and L235A (LALA), or
L234A, L235A and P329G (LALAPG), or
G236A, S239D and 1332E (GASDIE), or
G236A, S239D, A330L and 1332E (GASDALIE), or
S239D, H268F, S324T and 1332E (SDHFSTIE or SDH),
   the numbering being indicated in the sequence of a human IgG1 according to the EU index. Such mutations are in particular described in the Bruhns and Jonsson article, Immunol Rev. 2015 November; 268 (1): 25-51. Preferably, when the hybrid molecule is used in therapy, said mutated Fc fragment comprises at least the GASDIE, GASDALIE, or SDH mutations.

According to an embodiment, in said hybrid molecule according to the invention, said Fc fragment has a fucosylation level of between 0% and 100% of the glycosylated forms. According to the invention “between 0% and 100%” encompasses all integer values between 0 and 100, i.e.: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%. Low fucosylation of the Fc fragment elicits a strong ADCC response. This is why, according to a particular embodiment, said Fc fragment has a fucosylation level of between 0% and 60% of the glycosylated forms, in particular 50%, 40%, 30%, 20%, 10% or 0%. According to the invention, the fucosylation level is defined as the average proportion of fucose carried by the Fc fragment, relative to the maximum quantity of fucose that an Fc fragment can carry.

The Fc fragment and the peptide each have N- and C-terminal ends. The Fc fragment can therefore be bonded via its N- or C-terminal end to the N- or C-terminal end of the peptide. According to a preferred embodiment, in said hybrid molecule according to the invention, said covalent bond is located between the C-terminal end of said Fc fragment and the N-terminal end of said peptide, or between the N-terminal end of said Fc fragment and the N-terminal end of said peptide. According to an embodiment, when a spacer is present, the spacer can be bonded to the Fc fragment via its N- or C-terminal end. According to another embodiment, when two spacers are present, the first spacer can be bonded to the Fc fragment via its N- or C-terminal end and the second spacer can be bonded to the peptide via its N- or C-terminal end, in particular its N-terminal end. Alternatively, said covalent bond between said Fc fragment and said peptide (optionally in the presence of one or more spacers) can be created on all or part of the Fc fragment. According to the invention, “all or part of the Fc fragment” means that different amino acids constituting the Fc fragment may be involved in a covalent bond with said peptide.

According to a preferred embodiment, when at least one spacer is present in said hybrid molecule of the invention, this makes it possible to bond the Fc fragment to an azide or to an alkyne which will itself be involved in the covalent bond with said peptide. According to an embodiment, when at least one spacer is present in said hybrid molecule of the invention, the at least one spacer can also make it possible to bond the peptide to an alkyne or to an azide which will itself be involved in the covalent bond with the Fc fragment. According to a preferred embodiment, the hybrid molecule according to the invention comprises at least two spacers: a first spacer which makes it possible to bond the Fc fragment to an azide or to an alkyne, and a second spacer which makes it possible to bond the peptide to an azide (when the Fc fragment is bonded to an alkyne) or to an alkyne (when the Fc fragment is bonded to an azide).

According to an embodiment according to the invention, in said hybrid molecule according to the invention, said Fc fragment:
is coupled to at least one azide or an alkyne, such as a cyclooctyne, and in particular DBCO, or
is bonded to at least one spacer which is itself coupled to an azide or an alkyne, such as a cyclooctyne, and in particular DBCO, and said peptide is:
either coupled to an azide or to an alkyne, such as a cyclooctyne, and in particular DBCO,
either bonded to a spacer which is itself coupled to an azide or to an alkyne, such as a cyclooctyne, and in particular DBCO,
   the covalent bond between said Fc fragment and said peptide, optionally in the presence of one or more spacers, being created between the azide and the alkyne.

According to an embodiment according to the invention, in said hybrid molecule according to the invention,
the Fc fragment is coupled to at least one azide and said peptide is coupled to an alkyne, such as a cyclooctyne, and in particular DBCO, or
the Fc fragment is coupled to at least one alkyne, such as a cyclooctyne, and in particular DBCO, and said peptide is coupled to an azide, or
   the covalent bond between said Fc fragment and said peptide being created between the azide and the alkyne.

According to an embodiment according to the invention, in said hybrid molecule according to the invention:
the Fc fragment is coupled to at least one azide and said peptide is bonded to a spacer, itself coupled to an alkyne, such as a cyclooctyne, and in particular DBCO or
the Fc fragment is coupled to at least one alkyne, such as a cyclooctyne, and in particular DBCO, and said peptide is bonded to a spacer, itself coupled to an azide or
the Fc fragment is bonded to at least one spacer, itself coupled to an azide, and said peptide is coupled to an alkyne, such as a cyclooctyne, and in particular DBCO or
the Fc fragment is bonded to at least one spacer, itself coupled to an alkyne, such as a cyclooctyne, and in particular DBCO, and said peptide is coupled to an azide or
the Fc fragment is bonded to at least one spacer, itself coupled to an azide, and said peptide is bonded to a spacer, itself coupled to an alkyne, such as a cyclooctyne, and in particular DBCO or
the Fc fragment is bonded to at least one spacer, itself coupled to an alkyne, such as a cyclooctyne, and in particular DBCO, and said peptide is bonded to a spacer, itself coupled to an azide,
   the covalent bond between said Fc fragment and said peptide, in the presence of one or more spacers, being created between the azide and the alkyne.

According to the invention, the creation of the covalent bond between the azide and the alkyne corresponds to a step named a “click chemistry” step, wherein the N₃ part of the azide reacts with an alkyne. Azide means salts of hydrozoic acid HN₃, or organic azides in which one of the nitrogen atoms is covalently bonded to a carbon atom of an organic compound (e.g., methyl azide CH₃N₃). Preferably the azide is represented by the formula N₃. Alkynes are molecules with the general formula CₙH₂ₙ₋₂, which are characterized by the presence of at least one triple bond. Preferably the alkyne is a cyclooctyne, even more preferably dibenzocyclooctyne (DBCO).

The Fc fragment, said peptide and optionally said spacer(s), are coupled to the alkyne or to the azide by any conventionally used molecular coupling technique (such as conjugation). Any technique can also be used to covalently bond the Fc fragment to the spacer and/or the peptide to the spacer.

More specifically, a conjugation technique means an enzymatic conjugation or a chemical conjugation. An enzymatic conjugation means, for example, a conjugation using a transglutaminase which catalyzes the formation of covalent bonds between free amine groups and glutamine or lysine residues or using a transpeptidase such as sortase. For further information on enzymatic conjugation, see, e.g., patent application US20160361434 or US20170313787, or the publication by Ohtsuka et al., Bioscience, Biotechnology, and Biochemistry Volume 64, 2000-Issue 12, Comparison of Substrate Specificities of Transglutaminases Using Synthetic Peptides as Acyl donors. The substrate of the transglutaminase is for example a peptide comprising a glutamyl residue (a Qtag), as represented by SEQ ID NO: 34 (LLQG). A chemical conjugation means, for example, a covalent bond between an isolated cysteine or one part of a disulfide bridge after reduction of the latter and, for example, a maleimide. An example of such a conjugation is shown in FIG. 4. In this example, the Fc fragment comprises a Qtag peptide and said Fc fragment is bonded to a spacer (itself coupled to an azide), thanks to the action of transglutaminase which will create a covalent bond between the glutamyl residue of the Qtag and the N_{H2} group carried by the PEGn spacer.

According to the invention, the term “coupled” or “molecular coupling” means the establishment of a covalent bond, thus the Fc fragment and/or the peptide and/or the spacer is covalently bonded to an alkyne or an azide. The term “bonded” also applies to a covalent bond. Thus, by way of example, the expression “the Fc fragment is coupled to an azide and said peptide is bonded to a spacer, itself coupled to an alkyne, such as a cyclooctyne, and in particular DBCO” can also be read as “the Fc fragment is covalently bonded to an azide and said peptide is covalently bonded to a spacer, said spacer itself being covalently bonded to an alkyne, such as a cyclooctyne, and in particular DBCO”.

### Use of Hybrid Molecules According to the Invention

In a second aspect, the present invention also relates to a hybrid molecule as defined above, for its use as a drug.

More particularly, according to the invention, said hybrid molecules are intended to target and lyse in the body of patients, by ADCC and/or phagocytosis and/or complement activation, all the cells expressing autoreactive lymphocyte receptors within the framework of autoimmune dermatitis: namely the B-cells (lymphocytes) expressing on their surface the BCRs recognizing at least one of the peptides of the invention (autoantibodies responsible for autoimmune dermatitis, bonding to at least one peptide according to the invention) and T-lymphocytes expressing on their surface the TCRs recognizing at least one of the peptides according to the invention. Indeed, the hybrid molecule according to the invention bonds to these cells thanks to the peptide: this is the epitope target of said autoreactive lymphocyte receptor. The hybrid molecule according to the invention also bonds to cells making it possible to destroy B and/or T-lymphocytes expressing on their surface the autoreactive lymphocyte receptors involved in autoimmune dermatitis thanks to its Fc fragment, a natural ligand for Fc receptors (for example an Fc-gamma receptor (FcγR) if the Fc fragment comes from an IgG), present in particular on the surface of macrophages but also of NK-cells (“Natural Killers”).

According to a particular embodiment, the invention relates to a hybrid molecule, as defined above, for its use in the treatment of autoimmune dermatitis, in particular pemphigus or bullous pemphigoid. According to an embodiment, pemphigus is understood more particularly to mean pemphigus vulgaris or foliaceus.

According to a particular embodiment, the invention relates to a hybrid molecule as defined above and comprising a peptide selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, for its use in the treatment of bullous pemphigoid.

According to a particular embodiment, the invention relates to a hybrid molecule as defined above and comprising a peptide selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 38, SEQ ID NO: 39 and SEQ ID NO: 40, for its use in the treatment of bullous pemphigoid, preferably SEQ ID NO: 38 or SEQ ID NO: 40.

According to a particular embodiment, the invention relates to a hybrid molecule as defined above and comprising a peptide selected from the group consisting of: SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, for its use in the treatment of pemphigus, in particular pemphigus foliaceus.

According to a particular embodiment, the invention relates to a hybrid molecule as defined above and comprising a peptide selected from the group consisting of: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30, for its use in the treatment of pemphigus, in particular pemphigus vulgaris.

According to a particular embodiment, the invention relates to a hybrid molecule as defined above and comprising a peptide selected from the group consisting of: SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30, SEQ ID NO: 41, SEQ ID NO: 42, SEQ ID NO: 43, SEQ ID NO: 44, SEQ ID NO: 45, SEQ ID NO: 46, SEQ ID NO: 47 et SEQ ID NO: 48, for its use in the treatment of pemphigus, in particular pemphigus vulgaris. According to a particular embodiment, the invention relates to a hybrid molecule as defined above and comprising a peptide selected from the group consisting of: SEQ ID NO: 10, SEQ ID NO: 26, SEQ ID NO: 41, SEQ ID NO: 42, SEQ ID NO: 43, SEQ ID NO: 44, SEQ ID NO: 45, SEQ ID NO: 46, SEQ ID NO: 47 et SEQ ID NO: 48 for its use in the treatment of pemphigus vulgaris.

According to an embodiment, the invention also relates to a pharmaceutical composition comprising a hybrid molecule according to the invention, in combination with a pharmaceutically acceptable carrier.

According to the invention, “a pharmaceutically acceptable carrier” means any formulation making the composition suitable for its administration to a patient, in any galenic form.

The present invention also relates to a method for treating autoimmune dermatitis, in particular pemphigus or bullous pemphigoid, comprising administering a therapeutically effective amount of a hybrid molecule according to the invention.

The invention also relates to the use of a hybrid molecule according to the invention for preparing a drug intended for treating autoimmune dermatitis, in particular pemphigus or bullous pemphigoid.

According to another embodiment, the hybrid molecule according to the invention may be coupled to at least one radioisotope or to at least one fluorochrome, such as A488 or A647. Such molecules can advantageously be used as molecular tracer tools.

According to another embodiment, the invention thus relates to the in vitro or ex vivo use of a hybrid molecule comprising at least one antibody Fc fragment covalently bonded to at least one peptide bonding to an autoreactive lymphocyte involved in autoimmune dermatitis, at least one spacer being optionally present between said Fc fragment and said peptide, as a molecular tool. Such structures can be used in particular to analyze binding of hybrid molecules to the autoantibodies and to the Fc receptors of macrophages and NK cells, as well as to analyze the reactivity of macrophages and NK cells to binding the hybrids followed by bridging thereof by the autoantibodies . . . . Radioisotopes and/or fluorochromes are preferably coupled to the Fc fragment, even more particularly at the level of the Qtag (where available) or at the level of the lysines.

Method for producing the hybrid molecules according to the invention

In another aspect, the invention also relates to a method for obtaining a hybrid molecule as defined above.

According to an embodiment, the invention thus relates to a method for producing a hybrid molecule as defined above, comprising the following steps:
(ii) obtaining an azide coupled to an Fc fragment or obtaining an alkyne coupled to an Fc fragment, wherein
(ii) obtaining an alkyne coupled to a peptide or obtaining an azide coupled to a peptide, wherein
(iii) carrying out the covalent bond between the azide and the alkyne,
step (i) can be carried out before or after step (ii), or at the same time.

According to an embodiment, the invention thus relates to a method for producing a hybrid molecule as defined above, comprising the following steps:
(i) coupling at least one azide and one Fc fragment, optionally in the presence of at least one spacer, or coupling at least one alkyne and at least one Fc fragment, optionally in the presence of a spacer,
(ii) coupling an alkyne and a peptide, optionally in the presence of a spacer, or coupling an azide and a peptide, optionally in the presence of a spacer,
(iii) carrying out the covalent bond between the azide and the alkyne,
step (i) can be carried out before or after step (ii), or at the same time.

According to another embodiment, the invention thus relates to a method for producing a hybrid molecule as defined above, comprising the following steps:
(i) coupling at least one azide to each monomer of the Fc fragment, optionally in the presence of at least one spacer, or coupling at least one alkyne to each monomer of the Fc fragment, optionally in the presence of a spacer,
(ii) coupling an alkyne and a peptide, optionally in the presence of a spacer, or coupling an azide and a peptide, optionally in the presence of a spacer,
(iii) carrying out the covalent bond(s) between the azide and the alkyne,
step (i) can be carried out before or after step (ii), or at the same time.

According to an embodiment, the hybrid molecules according to the invention can also be obtained by bioproduction methods and encoded by a recombinant DNA encoding the peptide according to the invention and the Fc fragment, said peptide and said Fc fragment being optionally separated by a polypeptide linker. In such a case, the peptide of the hybrid molecule is unmodified (e.g., non-cyclized, non-retro, non-retro-inverso), and may be N-terminal or C-terminal oriented. The peptide may be at the C-terminal or N-terminal end of the Fc fragment, with the cDNA including its nucleotide coding sequence upstream or downstream of the cDNA of the Fc.

The sequences according to the invention are shown in Table 1 below.

TABLE 1
Summary table of the sequences according to the invention

| Sequence number/ | |
|---|---|
| Sequence name if | |
| applicable | Sequence |
| SEQ ID NO: 1 | RSILPYGDSMDRIEKDRLQGMAP |
| SEQ ID NO: 2 | CPCGSCCSWWKWLLGLLL |
| SEQ ID NO: 3 | EEVRKLKARVDELERIRRSILPYGDSMDRIEKDRLQGMAPAAGAD |
| SEQ ID NO: 4 | RSILPYGDSMDRIEKDRLQGMAPAAGADLDKIGLHSDSQEEL |
| SEQ ID NO: 5 | GLLFGLIALAEEVRKLKAR |
| SEQ ID NO: 6 | RSILPYGDSMDRIE |
| SEQ ID NO: 7 | SILPYGDSMDRIEKDRLQGMAPAAGADLDKIGLH |
| SEQ ID NO: 8 | DSQEELWMFVRKKLM |
| SEQ ID NO: 9 | TNVGILKVVKPLDYE |
| SEQ ID NO: 10 | ALNSMGQDLERPLELR |
| SEQ ID NO: 11 | QEPSDSPMFIINR |
| SEQ ID NO: 12 | RISGVGIDQPPYGIF |
| SEQ ID NO: 13 | PYGIFVINQKTGEIN |
| SEQ ID NO: 14 | FKIIRQEPSDSPMFI |
| SEQ ID NO: 15 | SPMFIINRNTGEIRT |
| SEQ ID NO: 16 | DREQYGQYALAVRGS |
| SEQ ID NO: 17 | AVRGSDRDGGADGMS |
| SEQ ID NO: 18 | FGNDDRTNTEPNTKI |
| SEQ ID NO: 19 | PNTKITTNTGRQEST |
| SEQ ID NO: 20 | RQESTSSTNYDTSTT |
| SEQ ID NO: 21 | ERPLELRVRVLDI |
| SEQ ID NO: 22 | REWVKFAKPCRE |
| SEQ ID NO: 23 | LNSKIAFKIVSQEPA |
| SEQ ID NO: 24 | DSQNNRCEMPRSLTLEVCQCDNRGICGTSYPTTSPGTRYGRPHSG |
| SEQ ID NO: 25 | NSKRNPIAKITSDYQ |
| SEQ ID NO: 26 | KVVKALDYEQLQSVK |
| SEQ ID NO: 27 | INVREGIAFRPASKT |
| SEQ ID NO: 28 | PASKTFTVQKGISSK |
| SEQ ID NO: 29 | FVKNMNRDSTFIVNK |
| SEQ ID NO: 30 | VKILDINDNPPVFSQQIFMGEIEENSASNSLVMILNATDADEPNHLNS |
| SEQ ID NO: 31/ | APELLGGPSVFLFPPKPKDTLMISRTPEVTCVVVDVSHEDPEVKFNWYVDGVEVHNAKT |
| Fc Fragment | KPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 32/ | LLQGARSDATHTCPPCPAPELLGGPSVFLFPPKPKDTLMISRTPEVTCVVVDVSHEDPE |
| Fc Fragment + | VKFNWYVDGVEVHNAKTKPREEQYNSTYRVVSVLTVLHQDWLNGKEYKCKVSNKALP |
| Qtag | APIEKTISKAKGQPREPQVYTLPPSREEMTKNQVSLTCLVKGFYPSDIAVEWESNGQPE |
| | NNYKTTPPVLDSDGSFFLYSKLTVDKSRWQQGNVFSCSVMHEALHNHYTQKSLSLSPG |
| | K |
| SEQ ID NO: 33/ | AHGHGHGLLQGARSDATHTCPPCPAPELLGGPSVFLFPPKPKDTLMISRTPEVTCVVVD |
| Fc Fragment + | VSHEDPEVKFNWYVDGVEVHNAKTKPREEQYNSTYRVVSVLTVLHQDWLNGKEYKCK |
| Qtag bis | VSNKALPAPIEKTISKAKGQPREPQVYTLPPSREEMTKNQVSLTCLVKGFYPSDIAVEWE |
| | SNGQPENNYKTTPPVLDSDGSFFLYSKLTVDKSRWQQGNVFSCSVMHEALHNHYTQK |
| | SLSLSPGK |
| SEQ ID NO: 34 | LLQG |
| (Qtag example) | |
| SEQ ID NO: 35/ | MDVTKKNKRDGTEVTERIVTETVTTRLTSLPPKGGTSNGYAKTASLGGGSRLEKQSLTH |
| α-1 polypeptide | GSSGYINSTGSTRGHASTSSYRRAHSPASTLPNSPGSTFERKTHVTRHAYEGSSSGNS |
| chain of collagen | SPEYPRKEFASSSTRGRSQTRESEIRVRLQSASPSTRWTELDDVKRLLKGSRSASVSPT |
| XVII | RNSSNTLPIPKKGTVETKIVTASSQSVSGTYDATILDANLPSHVWSSTLPAGSSMGTYHN |
| | NMTTQSSSLLNTNAYSAGSVFGVPNNMASCSPTLHPGLSTSSSVFGMQNNLAPSLTTL |
| | SHGTTTTSTAYGVKKNMPQSPAAVNTGVSTSAACTTSVQSDDLLHKDCKFLILEKDNTP |
| | AKKEMELLIMTKDSGKVFTASPASIAATSFSEDTLKKEKQAAYNADSGLKAEANGDLKTV |
| | STKGKTTTADIHSYGSSGGGGSGGGGGVGGAGGGPWGPAPAWCPCGSCCSWWWVKW |
| | LLGLLLTWLLLLGLLFGLIALAEEVRKLKARVDELERIRRSILPYGDSMDRIEKDRLQGMA |
| | PAAGADLDKIGLHSDSQEELWMFVRKKLMMEQENGNLRGSPGPKGDMGSPGPKGDR |
| | GFPGTPGIPGPLGHPGPQGPKGQKGSVGDPGMEGPMGQRGREGPMGPRGEAGPPG |
| | SGEKGERGAAGEPGPHGPPGVPGSVGPKGSSGSPGPQGPPGPVGLQGLRGEVGLPG |
| | VKGDKGPMGPPGPKGDQGEKGPRGLTGEPGMRGLPGAVGEPGAKGAMGPAGPDGH |
| | QGPRGEQGLTGMPGIRGPPGPSGDPGKPGLTGPQGPQGLPGTPGRPGIKGEPGAPG |
| | KIVTSEGSSMLTVPGPPGPPGAMGPPGPPGAPGPAGPAGLPGHQEVLNLQGPPGPPG |
| | PRGPPGPSIPGPPGPRGPPGEGLPGPPGPPGSFLSNSETFLSGPPGPPGPPGPKGDQ |
| | GPPGPRGHQGEQGLPGFSTSGSSSFGLNLQGPPGPPGPQGPKGDKGDPGVPGALGI |
| | PSGPSEGGSSSTMYVSGPPGPPGPPGPPGSISSSGQEIQQYISEYMQSDSIRSYLSGV |
| | QGPPGPPGPPGPVTTITGETFDYSELASHVVSYLRTSGYGVSLFSSSISSEDILAVLQRD |
| | DVRQYLRQYLMGPRGPPGPPGASGDGSLLSLDYAELSSRILSYMSSSGISIGLPGPPGP |
| | PGLPGTSYEELLSLLRGSEFRGIVGPPGPPGPPGIPGNVWSSISVEDLSSYLHTAGLSFI |
| | PGPPGPPGPPGPRGPPGVSGALATYAAENSDSFRSELISYLTSPDVRSFIVGPPGPPGP |
| | QGPPGDSRLLSTDASHSRGSSSSSHSSSVRRGSSYSSSMSTGGGGAGSLGAGGAFG |
| | EAAGDRGPYGTDIGPGGGYGAAAEGGMYAGNGGLLGADFAGDLDYNELAVRVSESM |
| | QRQGLLQGMAYTVQGPPGQPGPQGPPGISKVFSAYSNVTADLMDFFQTYGAIQGPPG |
| | QKGEMGTPGPKGDRGPAGPPGHPGPPGPRGHKGEKGDKGDQVYAGRRRRRSIAVKP |
| SEQ ID NO: 36/ | EWIKFAAACREGEDNSKRNPIAKIHSDCAANQQVTYRISGVGIDQPPYGIFVINQKTGEIN |
| Sequence of the | ITSIVDREVTPFFIIYCRALNSMGQDLERPLELRVRVLDINDNPPVFSMATFAGQIEENSN |
| DSG1 | ANTLVMILNATDADEPNNLNSKIAFKIIRQEPSDSPMFIINRNTGEIRTMNNFLDREQYGQ |
| | YALAVRGSDRDGGADGMSAECECNIKILDVNDNIPYMEQSSYTIEIQENTLNSNLLEIRVI |
| | DLDEEFSANWMAVIFFISGNEGNWFEIEMNERTNVGILKVVKPLDYEAMQSLQLSIGVR |
| | NKAEFHHSIMSQYKLKASAISVTVLNVIEGPVFRPGSKTYVVTGNMGSNDKVGDFVATD |
| | LDTGRPSTTVRYVMGNNPADLLAVDSRTGKLTLKNKVTKEQYNMLGGKYQGTILSIDDN |
| | LQRTCTGTININIQSFGNDDRTNTEPNTKITTNTGRQESTSSTNYDTSTTSTDSSQVYSS |
| | EPGNGAKDLLSDNVHFGPAGIGLLIMGFLVLGLVPFLMICCDCGGAPRSAAGFEPVPEC |
| | SDGAIHSWAVEGPQPEPRDITTVIPQIPPDNANIIECIDNSGVYTNEYGGREMQDLGGGE |
| | RMTGFELTEGVKTSGMPEICQEYSGTLRRNSMRECREGGLNMNFMESYFCQKAYAYA |
| | DEDEGRPSNDCLLIYDIEGVGSPAGSVGCCSFIGEDLDDSFLDTLGPKFKKLADISLGKE |
| | SYPDLDPSWPPQSTEPVCLPQETEPVVSGHPPISPHFGTTTVISESTYPSGPGVLHPKPI |
| | LDPLGYGNVTVTESYTTSDTLKPSVHVHDNRPASNVVVTERVVGPISGADLHGMLEMP |
| | DLRDGSNVIVTERVIAPSSSLPTSLTIHHPRESSNVVVTERVIQPTSGMIGSLSMHPELAN |
| | AHNVIVTERVVSGAGVTGISGTTGISGGIGSSGLVGTSMGAGSGALSGAGISGGGIGLSS |
| | LGGTASIGHMRSSSDHHFNQTIGSASPSTARSRITKYSTVQYSK |
| SEQ ID NO: 37/ | EWWVKFAKPCREGEDNSKRNPIAKITSDYQATQKITYRISGVGIDQPPFGIFVVDKNTGDIN |
| Sequence of the | ITAIVDREETPSFLITCRALNAQGLDVEKPLILTVKILDINDNPPVFSQQIFMGEIEENSASN |
| DSG3 | SLVMILNATDADEPNHLNSKIAFKIVSQEPAGTPMFLLSRNTGEVRTLTNSLDREQASSY |
| | RLVVSGADKDGEGLSTQCECNIKVKDVNDNFPMFRDSQYSARIEENILSSELLRFQVTD |
| | LDEEYTDNWLAVYFFTSGNEGNWFEIQTDPRTNEGILKVVKALDYEQLQSVKLSIAVKN |
| | KAEFHQSVISRYRVQSTPVTIQVINVREGIAFRPASKTFTVQKGISSKKLVDYILGTYQAID |
| | EDTNKAASNVKYVMGRNDGGYLMIDSKTAEIKFVKNMNRDSTFIVNKTITAEVLAIDEYT |
| | GKTSTGTVYVRVPDFNDNCPTAVLEKDAVCSSSPSVVVSARTLNNRYTGPYTFALEDQ |
| | PVKLPAVWSITTLNATSALLRAQEQIPPGVYHISLVLTDSQNNRCEMPRSLTLEVCQCDN |
| | RGICGTSYPTTSPGTRYGRPHSGRLGPAAIGLLLLGLLLLLLAPLLLLTCDCGAGSTGGV |
| | TGGFIPVPDGSEGTIHQWGIEGAHPEDKEITNICVPPVTANGADFMESSEVCTNTYARG |
| | TAVEGTSGMEMTTKLGAATESGGAAGFATGTVSGAASGFGAATGVGICSSGQSGTMR |
| | TRHSTGGTNKDYADGAISMNFLDSYFSQKAFACAEEDDGQEANDCLLIYDNEGADATG |
| | SPVGSVGCCSFIADDLDDSFLDSLGPKFKKLAEISLGVDGEGKEVQPPSKDSGYGIESC |
| | GHPIEVQQTGFVKCQTLSGSQGASALSTSGSVQPAVSIPDPLQHGNYLVTETYSASGSL |
| | VQPSTAGFDPLLTQNVIVTERVICPISSVPGNLAGPTQLRGSHTMLCTEDPCSRLI |
| SEQ ID NO: 38 | EEVRKLKARVDELERIRRS |
| SEQ ID NO: 39 | RIRRSILPYGDSMDRIEK |
| SEQ ID NO: 40 | MDRIEKDRLQGMAP |
| SEQ ID NO: 41 | YRISGVGIDQPPYGIFVINQKTGEIN |
| SEQ ID NO: 42 | RALNSMGQDLERPLELRVRVLDIN |
| SEQ ID NO: 43 | FKIIRQEPSDSPMFIINRNTGEIRT |
| SEQ ID NO: 44 | NERTNVGILKVVKPLDYE |
| SEQ ID NO: 45 | INVREGIAFRPASKTFTVQKGISSKK |
| SEQ ID NO: 46 | REGEDNSKRNPIAKITSDYQ |
| SEQ ID NO: 47 | GTSYPTTSPGTRYGRPHSG |
| SEQ ID NO: 48 | KFVKNMNRDSTFIVNK |

Other features, details and advantages of the invention will appear on reading the attached Figures and examples which illustrate the invention and are not intended to limit it in any way.

### BRIEF DESCRIPTION OF THE FIGURES

FIG. 1 shows the outline of an example of a hybrid molecule according to the invention.

A spacer (which is optional) is shown between the antibody Fc fragment and the peptide bonding to an autoreactive lymphocyte involved in autoimmune dermatitis (called “peptide according to the invention”).

FIG. 2 represents a B-lymphocyte expressing on its surface transmembrane autoantibodies/BCR bonded to a hybrid molecule according to the invention, said hybrid molecule itself being bonded by the Fc fragment to an NK-cell.

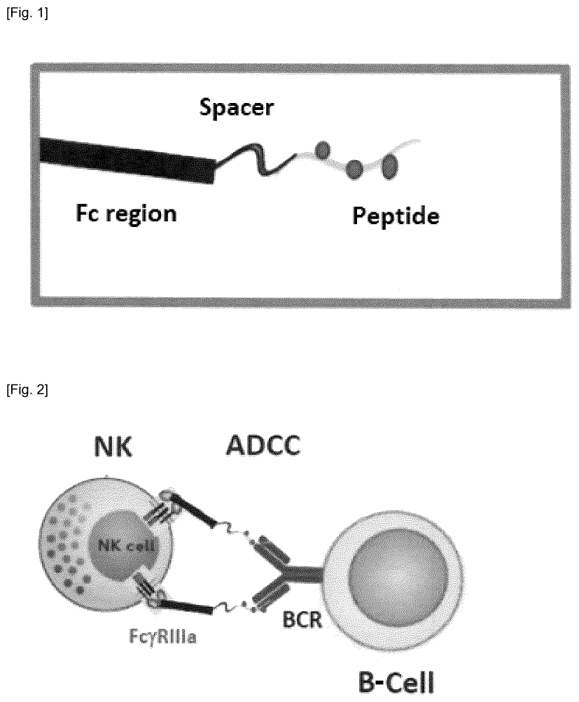

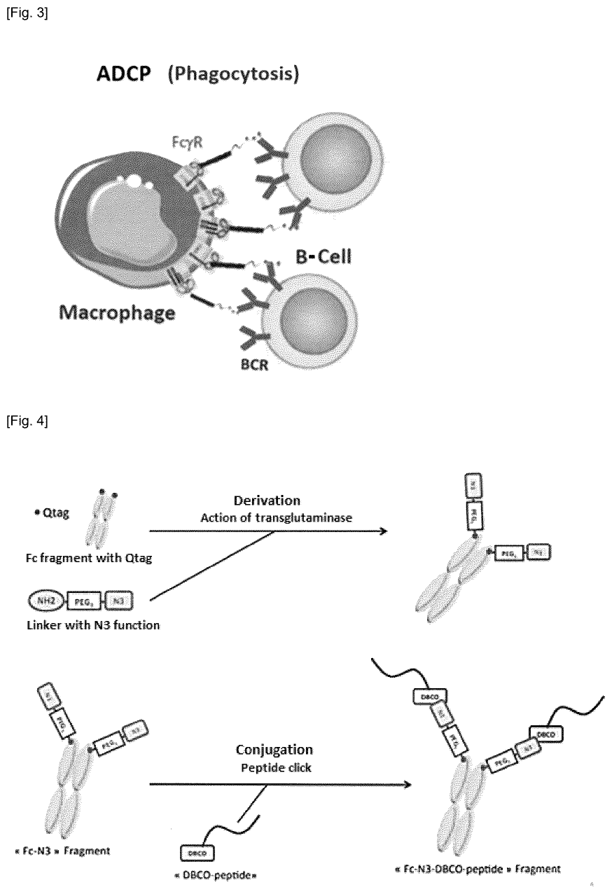

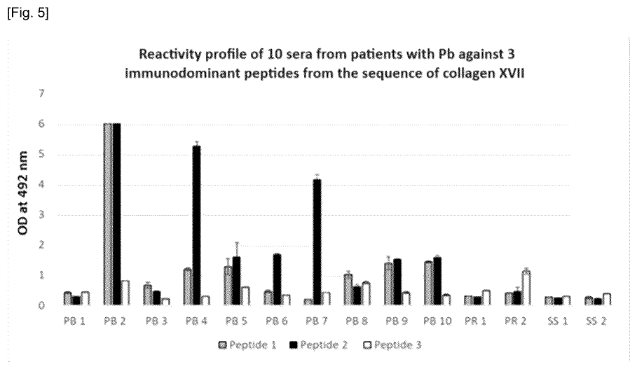

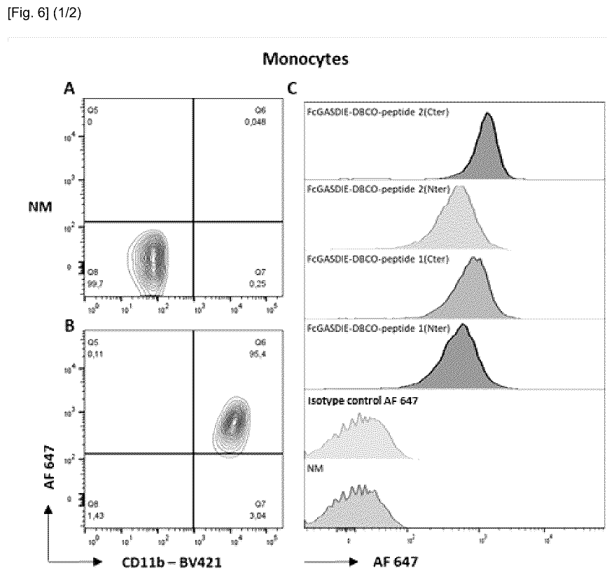

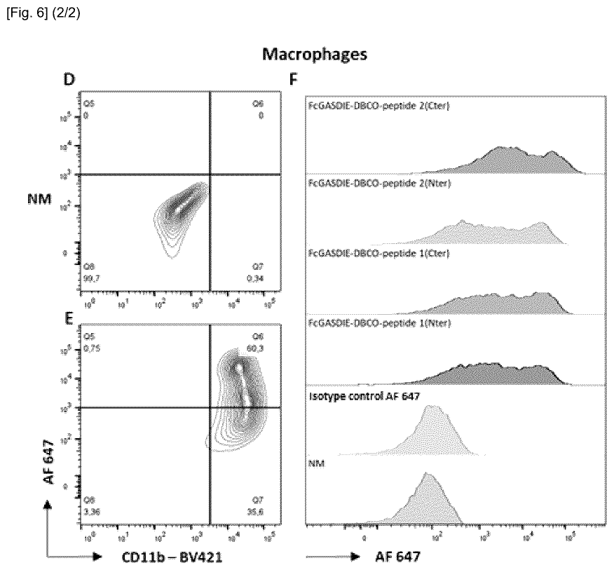

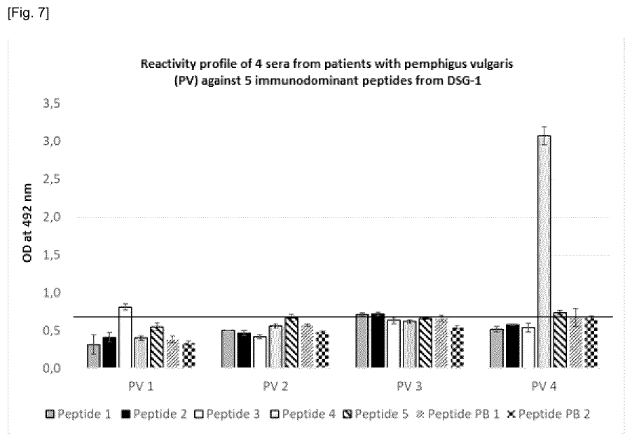

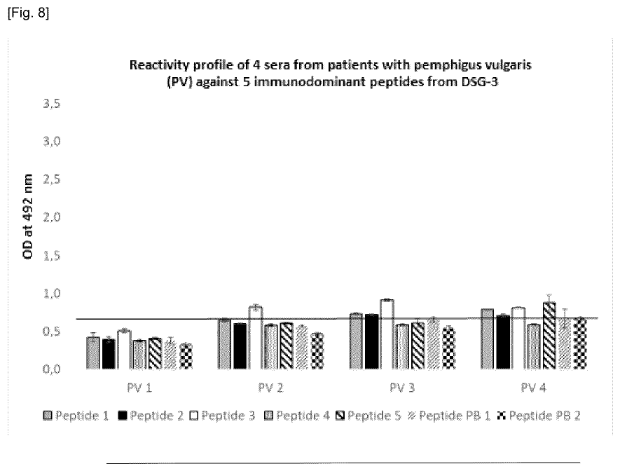

The NK-cell will thus be able to destroy the B-lymphocyte by ADCC. More specifically, this Figure shows a B-cell (or B-lymphocyte) which expresses on its surface a BCR (the autoantibody) which specifically interacts with the peptide according to the invention carried by the hybrid molecule. The connection of the Fc fragment carried by the same hybrid molecule to FcγRIIIa (CD16a) expressed on the surface of an NK-cell will activate ADCC and induce the specific destruction of the B-lymphocyte. (ADCC, Antibody-Dependent Cell Cytotoxicity; BCR, B-Cell Receptor; FcγR, Fc-gamma Receptor; NK cell, Natural Killer cell).

NB: a similar representation could be achieved by replacing the B-lymphocyte with a T-lymphocyte, and the BCR with a TCR (T-Cell Receptor).

FIG. 3 represents a B-lymphocyte expressing on its surface transmembrane BCR bonded to a hybrid molecule according to the invention, said hybrid molecule itself being bonded by the Fc fragment to a macrophage.

The macrophage will thus be able to destroy the B-lymphocyte by phagocytosis. More specifically, this Figure shows B-cells (or B-lymphocytes) which express on their surface a BCR (the autoantibody in membrane form) and which specifically interact with peptides according to the invention carried by the hybrid molecules. The engagement of Fc fragments carried by these same hybrid molecules to different FcγRs expressed on the surface of a macrophage will activate ADCP, i.e. phagocytosis, and induce the specific destruction of B-lymphocytes. (ADCP, Antibody-Dependent Cellular Phagocytosis; BCR, B-Cell Receptor; FcγR, Fc-gamma Receptor).

NB: a similar representation could be achieved by replacing the B-lymphocyte with a T-lymphocyte, and the BCR with a TCR (T-Cell Receptor).

FIG. 4 represents a method for manufacturing a hybrid molecule according to the invention, comprising the derivation and conjugation outline of the Fc fragments.

FIG. 5 represents the reactivity of 10 sera from patients with bullous pemphigoid (BP) with respect to three peptides from the polypeptide sequence of collagen XVII. Sera from patients with bullous pemphigoid contain autoantibodies.

These peptides include peptide 1 EEVRKLKARVDELERIRRS (SEQ ID NO: 38) whose sequence is included in the peptide SEQ ID NO: 3, peptide 2 RIRRSILPYGDSMDRIEK (SEQ ID NO: 39) whose sequence is included in the peptide SEQ ID NO: 3, is partly included in the peptide SEQ ID NO: 1, 4, 7 and the peptide SEQ ID NO: 6; finally peptide 3 MDRIEKDRLQGMAP (SEQ ID NO: 40) whose sequence is included in the peptides SEQ ID NO: 1, 3, 4 and 7 and partly included in the peptide SEQ ID NO: 1.

The ordinate represents the OD measured at 492 nm and the abscissa represents the tested serum. Sera PB1 to PB10 are sera collected from ten patients with bullous pemphigoid, sera PR1 and PR2 are sera from two patients with rheumatoid arthritis, and sera SS1 and SS2 are sera from two healthy patients.

FIG. 6 represents the binding of the hybrid molecules according to the invention on human monocytes and macrophages

Flow cytometry analysis of binding to the membrane of monocytes (FIG. 6C) and macrophages (FIG. 6F), of the 4 hybrid molecules FcGASDIE-DBCO-peptide 1(Nter), FcGASDIE-DBCO-peptide 1(Cter), FcGASDIE-DBCO-peptide 2(Nter) and FcGASDIE-DBCO-peptide 2(Cter) according to the invention, coupled to fluorochrome Alexa Fluor (AF) 647.

FcGASDIE-DBCO-peptide 1(Nter) corresponds to an Fc fragment comprising the G236A, S239D and 1332E mutations, which is bonded to at least one PEGn spacer which is itself coupled to an azide covalently bonded to a cyclooctyne DBCO coupled to a PEGn spacer which is itself bonded to the N-terminal end of peptide 1 EEVRKLKARVDELERIRRS (SEQ ID NO: 38).

FcGASDIE-DBCO-peptide 1(Cter) corresponds to an Fc fragment comprising the G236A, S239D and 1332E mutations, which is bonded to at least one PEGn spacer which is itself coupled to an azide covalently bonded to a cyclooctyne DBCO coupled to a PEGn spacer which is itself bonded to the C-terminal end of peptide 1 EEVRKLKARVDELERIRRS (SEQ ID NO: 38).

FcGASDIE-DBCO-peptide 2(Nter) corresponds to an Fc fragment comprising the G236A, S239D and 1332E mutations, which is bonded to at least one PEGn spacer which is itself coupled to an azide covalently bonded to a cyclooctyne DBCO coupled to a PEGn spacer which is itself bonded to the N-terminal end of peptide 2 RIRRSILPYGDSMDRIEK (SEQ ID NO: 39).

FcGASDIE-DBCO-peptide 2(Cter) corresponds to an Fc fragment comprising the G236A, S239D and 1332E mutations, which is bonded to at least one PEGn spacer which is itself coupled to an azide covalently bonded to a cyclooctyne DBCO coupled to a PEGn spacer which is itself bonded to the C-terminal end of peptide 2 RIRRSILPYGDSMDRIEK (SEQ ID NO: 39).

In section A, the Figure shows a flow cytometry diagram representing unlabeled monocytes (NM) as a scatter plot («density plot»). In section B, the Figure shows a representative result of monocyte labeling by an anti-CD11b antibody (AC BV421), the monocytes having also bound to the hybrid molecule FcGASDIE-DBCO-peptide 1(Nter) coupled to AF647. In section C, the Figure shows in the form of histograms, the binding profiles of the 4 hybrid molecules on the surface of monocytes, as well as that of the isotype control coupled with AF647 and that of the unlabeled monocytes (NM). In section D, the Figure shows a flow cytometry diagram representing unlabeled macrophages (NM) as a scatter plot («density plot»). In section E, the Figure shows a representative result of macrophage labeling by an anti-CD11b antibody (for example, antibody AC BV421), the macrophages having also bound to the hybrid molecule FcGASDIE-DBCO-peptide 1(Nter) coupled to AF647. In section F, the Figure shows in the form of histograms, the binding profiles of the 4 hybrid molecules on the surface of macrophages as well as that of the isotype control coupled with AF647 and that of the unlabeled macrophages (NM).

FIG. 7 represents the reactivity of 4 sera from patients with pemphigus vulgaris (PV) against 5 immunodominant peptides from desmoglein 1 (DSG-1). Sera from patients with pemphigoid vulgaris contain autoantibodies.

Peptides from desmoglein 1 include: peptide 1: YRISGVGIDQPPYGIFVINQKTGEIN (SEQ ID NO: 41) sequence included in that of SEQ ID NO: 36 and which includes peptide SEQ ID NO: 12 and the peptide SEQ ID NO: 13; peptide 2: RALNSMGQDLERPLELRVRVLDIN (SEQ ID NO: 42) sequence included in that of SEQ ID NO: 36 and which includes peptide SEQ ID NO: 10 and peptide SEQ ID NO: 21; peptide 3: FKIIRQEPSDSPMFIINRNTGEIRT (SEQ ID NO: 43) sequence included in that of SEQ ID NO: 36 and which includes peptide SEQ ID NO: 11, 14 and 15; peptide 4: NERTNVGILKVVKPLDYE (SEQ ID NO: 44) sequence included in that of SEQ ID NO: 36 and which includes peptide SEQ ID NO: 9 peptide 5: ALNSMGQDLERPLELR (SEQ ID NO: 10) sequence included in that of SEQ ID NO: 36.

The ordinate represents the OD measured at 492 nm and the abscissa represents the tested serum. Sera PV1 to PV4 represent sera collected from four patients with pemphigus vulgaris.

FIG. 8 represents the reactivity of 4 sera from patients with pemphigus vulgaris (PV) against 5 immunodominant peptides from desmoglein 3 (DSG-3). Sera from patients with pemphigoid vulgaris contain autoantibodies.

Peptides from desmoglein 3 include: peptide 1: INVREGIAFRPASKTFTVQKGISSKK (SEQ ID NO: 45), corresponding to an assembly covering INVREGIAFRPASKT SEQ ID NO: 27 and PASKTFTVQKGISSK SEQ ID NO: 28 sequences and to the sequence included in that of SEQ ID NO: 37; peptide 2: REGEDNSKRNPIAKITSDYQ (SEQ ID NO: 46) sequence included in that of SEQ ID NO: 27 and in that of SEQ ID NO: 37; peptide 3: GTSYPTTSPGTRYGRPHSG (SEQ ID NO: 47) sequence included in that of SEQ ID NO: 24 and in that of SEQ ID NO: 37; peptide 4: KVVKALDYEQLQSVK (SEQ ID NO: 26), sequence included in that of SEQ ID NO: 37 and peptide 5: KFVKNMNRDSTFIVNK (SEQ ID NO: 48): including peptide SEQ ID NO: 29); and sequence included in that of the peptide SEQ ID NO: 37.

The ordinate represents the OD measured at 492 nm and the abscissa represents the tested serum. Sera PV1 to PV4 represent sera collected from four patients with pemphigus vulgaris.

### EXAMPLES

### Example 1: Example of Production of a Hybrid Molecule According to the Invention

The hybrid molecule described here comprises the following structure: an Fc fragment covalently bonded to at least one PEGn spacer, itself coupled to an azide, said azide being covalently bonded to an alkyne, which is itself coupled to a PEGn spacer bonded to a peptide according to the invention. Such a molecule can be read: Fc-PEGn-N₃-DBCO-PEGn-peptide. The two PEGn may be the same or different (for example the first PEGn is PEG₃ and the second PEGn is PEG₂).

1. Synthesis of a NH2-PEGn-N₃ (i.e., a spacer coupled to an azide at one end and having a free amine function at another end).

2. Placement of NH₂-PEGn-N₃, with an Fc fragment having a Qtag, and a transglutaminase, preferably for 16 hours at 37° C. This so-called “derivation” step is, for example, carried out with 20 times more moles of NH₂-PEGn-N₃ than moles of Fc fragment. Transglutaminase is used, for example at a level of 15 U/μmol per present Qtag (on one or both monomers). Optionally, desalting may be carried out to remove excess spacer not bonded to the Fc fragment at the end of the step. A Fc-PEGn-N₃ is thus obtained. If the Fc fragment has 2Qtags (one carried on each monomer), then the Fc fragment can carry two PEGn-N₃.

3. Synthesis of a Cys-PEGn-peptide (i.e., a spacer bonded to a peptide at one end and having a free cysteine at another end). An alkyne, for example DBCO, is then coupled to the Cys-PEGn-peptide at the cysteine, and a DBCO-PEGn-peptide is thus obtained.

4. Achieving the “click”: coupling between the N₃ and the DBCO. The DBCO-PEGn-peptide is placed with the Fc-PEGn-N₃ with, preferably, 11 times more moles DBCO-PEGn-peptide than moles Fc-PEGn-N₃. The click reaction takes place in particular at room temperature and is almost complete after 4 hours.

5. Obtaining the hybrid molecule: Fc-PEGn-N₃-DBCO-PEGn-peptide. This embodiment is illustrated in FIG. 4.

Similarly, a Fc-PEGn-DBCO and a N₃-PEGn-peptide can be obtained, then coupled to obtain Fc-PEGn-DBCO-N₃-PEGn-peptide.

Similarly, a Fc-PEGn-DBCO and an N₃-peptide can be obtained, or a Fc-PEGn-N₃ and a DBCO-peptide, then coupled to obtain Fc-PEGn-DBCO-N₃-peptide or Fc-PEGn-N₃-DBCO-peptide, respectively.

### Example 2: Reactivity of Sera from Patients with Autoimmune Dermatitis to Hybrid Molecules According to the Invention

The sera used are sera from patients with autoimmune dermatitis, more particularly pemphigus or bullous pemphigoid, which contain specific autoantibodies.

ELISA microtiter plate wells were coated by passive adsorption using 100 μL of a solution containing either a peptide selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30, or a combination of at least two covalently bonded peptides, or a hybrid molecule according to the invention, wherein the Fc fragment is wild-type (WT) or the Fc fragment comprises the GASDIE mutation. Such a hybrid molecule is for example “FcWT-PEG-SEQ ID NO: 1-30”, “FcLALAPG-PEG-SEQ ID NO: 1-30” or “FcGASDIE-PEG-SEQ ID NO: 1-30». «FcWT-PEG-SEQ ID NO: 1-30” represents a wild-type Fc fragment which is bonded to at least one PEGn spacer which is itself coupled to an azide covalently bonded to a DBCO cyclooctyne coupled to a PEGn spacer which is itself bonded to a peptide represented by any one of SEQ ID NO: 1 to SEQ ID NO: 30, “FcWT-LALAPG-SEQ ID NO: 1-30” represents an Fc fragment, comprising the L234A, L235A and P329G mutations, which is bonded to at least one PEGn spacer which is itself coupled to an azide covalently bonded to a DBCO cyclooctyne coupled to a PEGn spacer which is itself bonded to a peptide represented by any one of SEQ ID NO: 1 to SEQ ID NO: 30, and “FcGASDIE-PEG-SEQ ID NO: 1-30” represents an Fc fragment, comprising the G236A, S239D and 1332E mutations, which is bonded to at least one PEGn spacer which is itself coupled to an azide covalently bonded to a DBCO cyclooctyne coupled to a PEGn spacer which is itself bonded to a peptide represented by any one of SEQ ID NO: 1 to SEQ ID NO: 30. In the structures, n represents a number between 1 and 10 when a PEGn spacer is used, preferably 1, 2, 3, 4 or 8. The hybrid molecule may contain one or a combination of at least 2 covalently bonded peptides.

These solutions were each used at a concentration of 5 μg/mL in a PBS (Phosphate Buffered Saline) buffer and incubated overnight at 4° C. The selected peptides and the hybrid molecules constructed with said peptides synthesized in scramble sequence (in French: “séquence aléatoire”) were used at the same concentration as negative controls. After washing in PBS Tween 0.05%, the wells were then saturated with 5% milk for 1 hour at room temperature. After washing, the sera were incubated in serial dilution in PBS buffer+5% milk for 1 hour at room temperature. After washing, the reactivity of the sera was detected using an anti-Fab detection antibody for detecting human IgG coupled with HRP diluted 1/2500 in PBS buffer 5% milk also incubated for 1 hour at room temperature. After washing, the presence of autoantibodies recognized by the hybrid molecule is revealed by adding TMB and stopping the enzymatic reaction by adding H2SO4 and reading on the spectrophotometer at 450 nm.

The results are expressed in Δ DO (Delta-Optical Density), corresponding to the DO obtained with a scramble hybrid peptide or with the hybrid molecule constructed with said peptide.

The results show a dose-dependent reactivity of sera on peptides with respect to hybrid molecules. They show that the peptides, when they are included in the different hybrids, remain perfectly reactive to the sera including the autoantibodies.

### Example 3: Binding Kinetics of the Hybrid Molecules onto Macrophages, at Physiological Temperature

Human macrophages, differentiated in vitro in the presence of M-CSF (100 ng/ml) from CD14+ monocytes isolated from peripheral blood of a healthy subject were incubated at 500,000 cells/well, in the presence of hybrid molecules at 5 μg/mL. Hybrid molecules are for example those described in Example 2.

The hybrid molecules are incubated for 2 h, 8 h, 16 h, 24 h and 48 h at 37° C. Binding of the hybrid molecules to the surface of macrophages is demonstrated and quantified by cytofluorimetry (FACS Canto II) after incubating the macrophages with an anti-Fc antibody coupled to FITC, used 1/1000.

“NM” and “ANTI FC” represent, respectively, the well containing macrophages incubated without antibody or with the sole anti-Fc antibody. “Wt”, “LALAPG” and “GASDIE” represent respectively the well containing the hybrid molecule with the wild-type Fc fragment or with the LALAPG mutation or with the GASDIE mutation.

The results show that the hybrid molecules at the physiological temperature of 37° C., bind to the membrane of macrophages. These results also show that the binding of the hybrid comprising an Fc fragment comprising the GASDIE mutation is greater than that of the wild form.

### Example 4: Phagocytosis Induced by the Fc-WT Hybrid or the Fc-GASDIE Hybrid when Arming B-Lymphocytes Via their FcγRIIb (CD32b) in the Presence of Sera

Freshly purified human B-lymphocytes from peripheral blood of healthy donors by CD19+magnetic sorting were labeled with green PKH-67 (Paul Karl Horan/fluorescent lipid marker), then incubated with sera from patients with autoimmune dermatitis at a concentration of 5 μg/mL for 30 to 60 min at 37° C. Subsequently, the cells were washed, then placed with the hybrid according to the invention comprising a wild-type Fc fragment (Wt) or comprising the GASDIE mutations. Hybrid molecules are for example those described in Example 2. The cells were placed in the presence of the hybrids at 10 g/mL for 30 to 60 min at 37° C. After washing, the cells were placed in contact with the macrophages (marked with red PKH-26), at a rate of 1 B-lymphocyte for 1 macrophage, for 2 hours at 37° C. The cells were then detached and analyzed by flow cytometry. Cells showing double fluorescence (green PKH-67/red PKH-26) correspond to macrophages that have phagocytosed lymphocytes. The different cell populations are expressed as a percentage of the total cell population: percentage of phagocytosis and percentage of B-lymphocytes.

The obtained results confirm the phagocytosis activity is increased in the presence of hybrids (increase in phagocytosis associated with a decrease in the B-lymphocyte population). Indeed, a significant increase in the percentage of phagocytosis, always associated with a significant decrease in the B-lymphocyte population, was observed when the B-lymphocytes were coated with the hybrid.

### Example 5: Binding Stability of Fc-GASDIE A488 and of the Fc-GASDIE Peptide Hybrids on NK-Cells after 30 Min, 24 h and 48 h, at Physiological Temperature

The NK cells were incubated for 30 min, 24 h or 48 h at 37° C. in the presence of FcGASDIE-N3-DBCO-A488 (an Fc fragment comprising the G236A, S239D and 1332E mutations which is bonded to at least one PEGn spacer which is itself coupled to an azide covalently bonded to a DBCO, the molecule being labeled with fluorochrome A488) or of FcGASDIE-N3-DBCO-PEG3-peptide-lysineA488 (an Fc fragment comprising the G236A, S239D and 1332E mutations which is bonded to at least one PEGn spacer which is itself coupled to an azide covalently bonded to a DBCO which is coupled to a PEGn spacer bonded to a peptide according to the invention (represented by any one of SEQ ID NOs: 1 to SEQ ID NO: 30) the molecule being labeled with fluorochrome A488 which is bonded to the molecule via a lysine). Binding of these 2 fluorescent probes to NK cells was analyzed by cytofluorimetry. In the structures, n represents a number between 1 and 10 when a PEGn spacer is used, preferably 1, 2, 3, 4 or 8.

Results are shown at 30 minutes, 24 hours and 48 hours. NM represents unlabeled NK-cells, without antibodies.

### Example 6: Reactivity of 10 Sera from Patients with Bullous Pemphigoid (BP) to 3 Immunodominant Peptides from the Polypeptide Sequence of Collagen XVII

ELISA plate wells were coated with 100 μL of a solution containing the peptide EEVRKLKARVDELERIRRS (represented by SEQ ID NO: 38, sequence included in the peptide SEQ ID NO: 3, also called peptide 1) or the peptide RIRRSILPYGDSMDRIEK (represented by SEQ ID NO: 39, sequence included in the peptide SEQ ID NO: 3, is partly included in the peptide SEQ ID NO: 1, 4, 7 and included in peptide SEQ ID NO: 6, also called peptide 2) or the peptide MDRIEKDRLQGMAP (represented by SEQ ID NO: 40, sequence included in the peptides SEQ ID NO: 1, 3, 4 and 7 and partly included in the peptide SEQ ID NO: 1, also called peptide 3), at a concentration of 10 μg/mL in a 0.15M NaCl PBS (Phosphate Buffered Saline) buffer, incubated overnight at 4° C.

The wells were then saturated with 2% BSA (Bovine Serum Albumin) in PBS buffer with 0.15M NaCl for 1 hour at 4° C. After washing, the 10 sera from BP patients, 2 sera from patients with rheumatoid arthritis (named PR1 and PR2) and 2 sera from healthy subjects (named SS1 and SS2) were incubated diluted 1/100 in PBS 2% BSA 2M NaCl buffer for 1 hour at 4° C. After washing, the reactivity of the sera was detected using a secondary goat anti-human IgG antibody coupled to peroxidase (HRP: Horse Radish Peroxidase) diluted 1/2500 in PBS 2% BSA buffer. The results are expressed in OD (Optical Density), read at 492 nm. The horizontal line represents the significance threshold.

The results shown in FIG. 5 show that 7 out of the 10 sera from patients with PB exhibit significant reactivity of very variable intensity to peptide 2, with three of them exhibiting very strong reactivity, whereas 5 out of the 10 exhibit significant but weaker reactivity to peptide 1 and only 2 out of the 10 exhibit very weak reactivity to peptide 3. There is significant interindividual heterogeneity of reactivity and heterogeneity of reactivity for a given patient with respect to the 3 peptides. As expected, the sera used as controls (PR1, PR2, SS1 and SS2) do not show significant reactivity towards the tested peptides, with the exception of a weak reactivity of one of the patients with rheumatoid arthritis towards peptide 3.

### Example 7: Binding of the Hybrid Molecules According to the Invention on Human Monocytes and Macrophages

CD14+ monocytes isolated from the peripheral blood of a healthy subject and macrophages, differentiated in vitro in the presence of M-CSF (100 ng/ml) from these same monocytes, were incubated at 100,000 cells/well for 1 h at 37° C., in the presence of the following hybrid molecules at 10 ug/mL:
FcGASDIE-DBCO-peptide 1(Nter) (an Fc fragment comprising the G236A, S239D and 1332E mutations, which is bonded to at least one PEGn spacer which is itself coupled to an azide covalently bonded to a cyclooctyne DBCO coupled to a PEGn spacer which is itself bonded to the N-terminal end of peptide 1 EEVRKLKARVDELERIRRS (SEQ ID NO: 38)),
FcGASDIE-DBCO-peptide 1(Cter) (an Fc fragment comprising the G236A, S239D and 1332E mutations, which is bonded to at least one PEGn spacer which is itself coupled to an azide covalently bonded to a cyclooctyne DBCO coupled to a PEGn spacer which is itself bonded to the C-terminal end of peptide 1 EEVRKLKARVDELERIRRS (SEQ ID NO: 38)),
FcGASDIE-DBCO-peptide 2(Nter) (an Fc fragment comprising the G236A, S239D and 1332E mutations, which is bonded to at least one PEGn spacer which is itself coupled to an azide covalently bonded to a cyclooctyne DBCO coupled to a PEGn spacer which is itself bonded to the N-terminal end of peptide 2 RIRRSILPYGDSMDRIEK (SEQ ID NO: 39)),
FcGASDIE-DBCO-peptide 2(Cter) (an Fc fragment comprising the G236A, S239D and 1332E mutations, which is bonded to at least one PEGn spacer which is itself coupled to an azide covalently bonded to a cyclooctyne DBCO coupled to a PEGn spacer which is itself bonded to the C-terminal end of peptide 2 RIRRSILPYGDSMDRIEK (SEQ ID NO: 39)).

These 4 hybrid molecules according to the invention, FcGASDIE-DBCO-peptide 1(Nter), FcGASDIE-DBCO-peptide 1(Cter), FcGASDIE-DBCO-peptide 2(Nter), FcGASDIE-DBCO-peptide 2(Cter), were labeled with an Alexa Fluor 647 fluorochrome (Kit “Lightning link”; Abcam). Binding of the hybrid molecules labeled with fluorochrome AF647 to the surface of monocytes and macrophages is demonstrated by cytofluorimetry (FACS Canto II). Over 10,000 events were analyzed per condition. The “NM” and “AF647 isotype control” conditions respectively represent the wells containing monocytes or macrophages incubated without antibody or with a non-specific antibody corresponding to an isotype control coupled to the Alexa Fluor (AF) 647 fluorochrome. CD11b-BV421 labeling allows monocytes and macrophages to be highlighted.

The results shown in FIG. 6 and in Table 2 below show that, at the physiological temperature of 37° C., the four hybrid molecules according to the invention bind to the membrane of monocytes and macrophages already from the first hour on. More than 90% of monocytes and more than 55% of macrophages bind with the hybrid molecules. For each hybrid molecule, the labeling heterogeneity on the surface of macrophages is greater than that observed on the surface of monocytes. It is noted that the FcGASDIE-DBCO-peptide 2(Cter) hybrid molecule presents a more intense labeling than that of the 3 other hybrid molecules (histogram shift towards high values).

TABLE 2
Binding results of hybrid molecules on the surface
of monocytes and macrophages at 37° C.

| | Monocytes | | Macrophages | |
|---|---|---|---|---|
| | MFI | Ratio | MFI | Ratio |
| Isotype control | 265 | 1 | 1638 | 1 |
| FcGASDIE-DBCO-peptide 1 (Nter) | 516 | 1.9 | 7388 | 4.5 |
| FcGASDIE-DBCO-peptide 1 (Cter) | 744 | 2.8 | 9129 | 5.6 |
| FcGASDIE-DBCO-peptide 2 (Nter) | 477 | 1.8 | 8042 | 4.9 |
| FcGASDIE-DBCO-peptide 2 (Cter) | 1228 | 4.6 | 10669 | 6.5 |

MFI: Mean Fluorescence Intensity; Ratio: ratio between the MFI obtained with each hybrid molecule and that obtained with the isotype control.

### Example 8: Reactivity of 4 Sera from Patients with Pemphigus Vulgaris (PV) to 5 Immunodominant Peptides from DSG-1

ELISA plate wells were coated with 100 μL of a solution containing a peptide from desmoglein 1:

-peptide 1:
(SEQ ID NO: 41)
YRISGVGIDQPPYGIFVINQKTGEIN,
or
-peptide 2:
(SEQ ID NO: 42)
RALNSMGQDLERPLELRVRVLDIN,
or
-peptide 3:
(SEQ ID NO: 43)
FKIIRQEPSDSPMFIINRNTGEIRT,
or
-peptide 4:
(SEQ ID NO: 44)
NERTNVGILKVVKPLDYE,
or
-peptide 5:
(SEQ ID NO: 10)
ALNSMGQDLERPLELR.

or a control peptide from collagen XVII:

-peptide 1:
(SEQ ID NO: 38, also called peptide PB1 in FIG. 7)
EEVRKLKARVDELERIRRS,
or
-peptide 2:
(SEQ ID NO: 39, also called peptide PB2 in FIG 7)
RIRRSILPYGDSMDRIEK.

The peptides are incubated at a concentration of 0 μg/mL in a 0.15M NaCl PBS (Phosphate Buffered Saline) buffer, overnight at 4° C.

The wells were then saturated with 2% BSA (Bovine Serum Albumin) in PBS 0.15M NaCl buffer for 1 hour at 4° C. After washing, the 4 sera from Pg patients were incubated diluted 1/100 in PBS 2% BSA 2M NaCl buffer for 1 hour at 4° C. After washing, the reactivity of the sera was detected using a secondary goat anti-human IgG antibody coupled to peroxidase (HRP: Horse Radish Peroxidase) diluted 1/2500 in PBS 2% BSA buffer. The results are expressed in OD (Optical Density), read at 492 nm. The horizontal line represents the significance threshold.

The results shown in FIG. 7 show that only serum PV3 shows a weak reactivity towards peptide 1 and peptide 2, that only serum PV1 shows clear reactivity towards peptide 3, that only serum PV5 very strongly recognizes peptide 4, and significantly recognizes peptide 5. As expected, none of the 4 sera significantly recognizes the 2 peptides PB1 and PB2, targets of the autoantibodies associated with bullous pemphigoid.

### Example 9: Reactivity of 4 Sera from Patients with Pemphigus Vulgaris (PV) to 5 Immunodominant Peptides from DSG-3

ELISA plate wells were coated with 100 μL of a solution containing a peptide from desmoglein 3:

-peptide 1:
(SEQ ID NO: 45)
INVREGIAFRPASKTFTVQKGISSKK;
-peptide 2:
(SEQ ID NO: 46)
REGEDNSKRNPIAKITSDYQ;
-peptide 3:
(SEQ ID NO: 47)
GTSYPTTSPGTRYGRPHSG;
-peptide 4:
(SEQ ID NO: 26)
KVVKALDYEQLQSVK,
and
-peptide 5:
(SEQ ID NO: 48)
KFVKNMNRDSTFIVNK,

or a control peptide from collagen XVII:

-peptide 1
(SEQ ID NO: 38, also called peptide PB1 in FIG. 8)
EEVRKLKARVDELERIRRS,
or
-peptide 2
(SEQ ID NO: 39, also called peptide PB2 in FIG. 8)
RIRRSILPYGDSMDRIEK.

The peptides are incubated at a concentration of 0 μg/mL in a 0.15M NaCl PBS (Phosphate Buffered Saline) buffer, overnight at 4° C.

The wells were then saturated with 2% BSA (Bovine Serum Albumin) in PBS 0.15M NaCl buffer for 1 hour at 4° C. After washing, the 4 sera from Pg patients were incubated diluted 1/100 in PBS 2% BSA 2M NaCl buffer for 1 hour at 4° C. After washing, the reactivity of the sera was detected using a secondary goat anti-human IgG antibody coupled to peroxidase (HRP: Horse Radish Peroxidase) diluted 1/2500 in PBS 2% BSA buffer. The results are expressed in OD (Optical Density), read at 492 nm. The horizontal line represents the significance threshold.

The results shown in FIG. 8 show that only sera PV3 and PV4 show weak reactivity to peptide 1 and peptide 2, that only sera PV2, PV3 and PV4 show clear reactivity to peptide 3, that none of the sera recognize peptide 4, and finally that only serum PV4 noticeably recognizes peptide 5. As expected, none of the 4 sera significantly recognizes the 2 peptides PB1 and PB2, targets of the autoantibodies associated with bullous pemphigoid.

1. A hybrid molecule comprising at least one antibody Fc fragment covalently bonded to at least one peptide bonding to an autoreactive lymphocyte involved in autoimmune dermatitis, at least one spacer being optionally present between said Fc fragment and said peptide.

2. A hybrid molecule according to claim 1, wherein said peptide comprises all or part of the sequence of desmoglein 1, desmoglein 3, and/or type XVII collagen.

3. A hybrid molecule according to claim 1, wherein the sequence of desmoglein 1, desmoglein 3, or type XVII collagen is of human origin.

4. A hybrid molecule according to claim 1, wherein said spacer is a polymer containing one or more repeating units containing the ether group.

5. A hybrid molecule according to claim 1, wherein said peptide is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 et SEQ ID NO: 48.

6. A hybrid molecule according to claim 1, wherein said Fc fragment is a human Fc fragment.

7. A hybrid molecule according to claim 1, wherein said Fc fragment is a wild-type or mutated fragment, said mutated Fc fragment preferably comprising at least the following mutations:
L234A and L235A, or
L234A, L235A and P329G, or
G236A, S239D and 1332E, or
G236A, S239D, A330L and 1332E, or
S239D, H268F, S324T and 1332E,
the numbering being indicated in the sequence of a human IgG1 according to the EU index.

8. A hybrid molecule according to claim 1, in which:
the Fc fragment is coupled to at least one azide and said peptide is coupled to an alkyne, or
the Fc fragment is coupled to at least one alkyne, and said peptide is coupled to an azide, or
the Fc fragment is coupled to at least one azide and said peptide is bonded to a spacer itself coupled to an alkyne, or
the Fc fragment is coupled to at least one alkyne, and said peptide is bonded to a spacer itself coupled to an azide or
the Fc fragment is bonded to at least one spacer, itself coupled to an azide, and said peptide is coupled to an alkyne, or
the Fc fragment is bonded to at least one spacer, itself coupled to an alkyne, and said peptide is coupled to an azide or
the Fc fragment is bonded to at least one spacer, itself coupled to an azide, and said peptide is bonded to a spacer, itself coupled to an alkyne, or
the Fc fragment is bonded to at least one spacer, itself coupled to an alkyne, and said peptide is bonded to a spacer, itself coupled to an azide,
wherein the covalent bond between said Fc fragment and said peptide, optionally in the presence of one or more spacers, is created between the azide and the alkyne.

9. A method of treatment of autoimmune dermatitis comprising administering a therapeutically effective amount of a hybrid molecule according to claim 1.

10. A hybrid molecule according to claim 2 in which said peptide comprises the α-1 chain of type XVII collagen.

11. A hybrid molecule according to claim 4 in which said spacer is polyethylene glycol of formula PEGn, wherein n denotes an integer between 1 and 100.

12. A hybrid molecule according to claim 11 in which said spacer is polyethylene glycol of formula PEGn, wherein n denotes an integer 1, 2, 3, 4 or 8.

13. A hybrid molecule according to claim 1 in which said Fc fragment is a human Fc of IgG.

14. A hybrid molecule according to claim 1 in which said Fc fragment is a human Fc of IgG1.

15. A hybrid molecule according to claim 8 in which said alkyne is a cyclooctyne.

16. A hybrid molecule according to claim 15 in which said cyclooctyne is DBCO.

17. A method of treatment according to claim 9, wherein said autoimmune dermatitis is pemphigus vulgaris or bullous pemphigoid.


SEQUENCE LISTING (48 sequences)

SEQ ID NO: 1 - protein, 23 aa, Homo sapiens
RSILPYGDSMDRIEKDRLQGMAP

SEQ ID NO: 2 - protein, 18 aa, Homo sapiens
CPCGSCCSWWKWLLGLLL

SEQ ID NO: 3 - protein, 45 aa, Homo sapiens
EEVRKLKARVDELERIRRSILPYGDSMDRIEKDRLQGMAPAAGAD

SEQ ID NO: 4 - protein, 42 aa, Homo sapiens
RSILPYGDSMDRIEKDRLQGMAPAAGADLDKIGLHSDSQEEL

SEQ ID NO: 5 - protein, 19 aa, Homo sapiens
GLLFGLIALAEEVRKLKAR

SEQ ID NO: 6 - protein, 14 aa, Homo sapiens
RSILPYGDSMDRIE

SEQ ID NO: 7 - protein, 34 aa, Homo sapiens
SILPYGDSMDRIEKDRLQGMAPAAGADLDKIGLH

SEQ ID NO: 8 - protein, 15 aa, Homo sapiens
DSQEELWMFVRKKLM

SEQ ID NO: 9 - protein, 15 aa, Homo sapiens
TNVGILKVVKPLDYE

SEQ ID NO: 10 - protein, 16 aa, Homo sapiens
ALNSMGQDLERPLELR

SEQ ID NO: 11 - protein, 13 aa, Homo sapiens
QEPSDSPMFIINR

SEQ ID NO: 12 - protein, 15 aa, Homo sapiens
RISGVGIDQPPYGIF

SEQ ID NO: 13 - protein, 15 aa, Homo sapiens
PYGIFVINQKTGEIN

SEQ ID NO: 14 - protein, 15 aa, Homo sapiens
FKIIRQEPSDSPMFI

SEQ ID NO: 15 - protein, 15 aa, Homo sapiens
SPMFIINRNTGEIRT

SEQ ID NO: 16 - protein, 15 aa, Homo sapiens
DREQYGQYALAVRGS

SEQ ID NO: 17 - protein, 15 aa, Homo sapiens
AVRGSDRDGGADGMS

SEQ ID NO: 18 - protein, 15 aa, Homo sapiens
FGNDDRTNTEPNTKI

SEQ ID NO: 19 - protein, 15 aa, Homo sapiens
PNTKITTNTGRQEST

SEQ ID NO: 20 - protein, 15 aa, Homo sapiens
RQESTSSTNYDTSTT

SEQ ID NO: 21 - protein, 13 aa, Homo sapiens
ERPLELRVRVLDI

SEQ ID NO: 22 - protein, 12 aa, Homo sapiens
REWVKFAKPCRE

SEQ ID NO: 23 - protein, 15 aa, Homo sapiens
LNSKIAFKIVSQEPA

SEQ ID NO: 24 - protein, 45 aa, Homo sapiens
DSQNNRCEMPRSLTLEVCQCDNRGICGTSYPTTSPGTRYGRPHSG

SEQ ID NO: 25 - protein, 15 aa, Homo sapiens
NSKRNPIAKITSDYQ

SEQ ID NO: 26 - protein, 15 aa, Homo sapiens
KVVKALDYEQLQSVK

SEQ ID NO: 27 - protein, 15 aa, Homo sapiens
INVREGIAFRPASKT

SEQ ID NO: 28 - protein, 15 aa, Homo sapiens
PASKTFTVQKGISSK

SEQ ID NO: 29 - protein, 15 aa, Homo sapiens
FVKNMNRDSTFIVNK

SEQ ID NO: 30 - protein, 48 aa, Homo sapiens
VKILDINDNPPVFSQQIFMGEIEENSASNSLVMILNATDADEPNHLNS

SEQ ID NO: 31 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 234 aa, synthetic construct
LLQGARSDATHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 241 aa, synthetic construct
AHGHGHGLLQGARSDATHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 4 aa, synthetic construct
LLQG

SEQ ID NO: 35 - protein, 1497 aa, Homo sapiens
MDVTKKNKRDGTEVTERIVTETVTTRLTSLPPKGGTSNGYAKTASLGGGSRLEKQSLTHGSSGYINSTGSTRGHASTSSYRRAHSPASTLPNSPGSTFERKTHVTRHAYEGSSSGNSSPEYPRKEFASSSTRGRSQTRESEIRVRLQSASPSTRWTELDDVKRLLKGSRSASVSPTRNSSNTLPIPKKGTVETKIVTASSQSVSGTYDATILDANLPSHVWSSTLPAGSSMGTYHNNMTTQSSSLLNTNAYSAGSVFGVPNNMASCSPTLHPGLSTSSSVFGMQNNLAPSLTTLSHGTTTTSTAYGVKKNMPQSPAAVNTGVSTSAACTTSVQSDDLLHKDCKFLILEKDNTPAKKEMELLIMTKDSGKVFTASPASIAATSFSEDTLKKEKQAAYNADSGLKAEANGDLKTVSTKGKTTTADIHSYGSSGGGGSGGGGGVGGAGGGPWGPAPAWCPCGSCCSWWKWLLGLLLTWLLLLGLLFGLIALAEEVRKLKARVDELERIRRSILPYGDSMDRIEKDRLQGMAPAAGADLDKIGLHSDSQEELWMFVRKKLMMEQENGNLRGSPGPKGDMGSPGPKGDRGFPGTPGIPGPLGHPGPQGPKGQKGSVGDPGMEGPMGQRGREGPMGPRGEAGPPGSGEKGERGAAGEPGPHGPPGVPGSVGPKGSSGSPGPQGPPGPVGLQGLRGEVGLPGVKGDKGPMGPPGPKGDQGEKGPRGLTGEPGMRGLPGAVGEPGAKGAMGPAGPDGHQGPRGEQGLTGMPGIRGPPGPSGDPGKPGLTGPQGPQGLPGTPGRPGIKGEPGAPGKIVTSEGSSMLTVPGPPGPPGAMGPPGPPGAPGPAGPAGLPGHQEVLNLQGPPGPPGPRGPPGPSIPGPPGPRGPPGEGLPGPPGPPGSFLSNSETFLSGPPGPPGPPGPKGDQGPPGPRGHQGEQGLPGFSTSGSSSFGLNLQGPPGPPGPQGPKGDKGDPGVPGALGIPSGPSEGGSSSTMYVSGPPGPPGPPGPPGSISSSGQEIQQYISEYMQSDSIRSYLSGVQGPPGPPGPPGPVTTITGETFDYSELASHVVSYLRTSGYGVSLFSSSISSEDILAVLQRDDVRQYLRQYLMGPRGPPGPPGASGDGSLLSLDYAELSSRILSYMSSSGISIGLPGPPGPPGLPGTSYEELLSLLRGSEFRGIVGPPGPPGPPGIPGNVWSSISVEDLSSYLHTAGLSFIPGPPGPPGPPGPRGPPGVSGALATYAAENSDSFRSELISYLTSPDVRSFIVGPPGPPGPQGPPGDSRLLSTDASHSRGSSSSSHSSSVRRGSSYSSSMSTGGGGAGSLGAGGAFGEAAGDRGPYGTDIGPGGGYGAAAEGGMYAGNGGLLGADFAGDLDYNELAVRVSESMQRQGLLQGMAYTVQGPPGQPGPQGPPGISKVFSAYSNVTADLMDFFQTYGAIQGPPGQKGEMGTPGPKGDRGPAGPPGHPGPPGPRGHKGEKGDKGDQVYAGRRRRRSIAVKP

SEQ ID NO: 36 - protein, 1000 aa, Homo sapiens
EWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNTKITTNTGRQESTSSTNYDTSTTSTDSSQVYSSEPGNGAKDLLSDNVHFGPAGIGLLIMGFLVLGLVPFLMICCDCGGAPRSAAGFEPVPECSDGAIHSWAVEGPQPEPRDITTVIPQIPPDNANIIECIDNSGVYTNEYGGREMQDLGGGERMTGFELTEGVKTSGMPEICQEYSGTLRRNSMRECREGGLNMNFMESYFCQKAYAYADEDEGRPSNDCLLIYDIEGVGSPAGSVGCCSFIGEDLDDSFLDTLGPKFKKLADISLGKESYPDLDPSWPPQSTEPVCLPQETEPVVSGHPPISPHFGTTTVISESTYPSGPGVLHPKPILDPLGYGNVTVTESYTTSDTLKPSVHVHDNRPASNVVVTERVVGPISGADLHGMLEMPDLRDGSNVIVTERVIAPSSSLPTSLTIHHPRESSNVVVTERVIQPTSGMIGSLSMHPELANAHNVIVTERVVSGAGVTGISGTTGISGGIGSSGLVGTSMGAGSGALSGAGISGGGIGLSSLGGTASIGHMRSSSDHHFNQTIGSASPSTARSRITKYSTVQYSK

SEQ ID NO: 37 - protein, 950 aa, Homo sapiens
EWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKDAVCSSSPSVVVSARTLNNRYTGPYTFALEDQPVKLPAVWSITTLNATSALLRAQEQIPPGVYHISLVLTDSQNNRCEMPRSLTLEVCQCDNRGICGTSYPTTSPGTRYGRPHSGRLGPAAIGLLLLGLLLLLLAPLLLLTCDCGAGSTGGVTGGFIPVPDGSEGTIHQWGIEGAHPEDKEITNICVPPVTANGADFMESSEVCTNTYARGTAVEGTSGMEMTTKLGAATESGGAAGFATGTVSGAASGFGAATGVGICSSGQSGTMRTRHSTGGTNKDYADGAISMNFLDSYFSQKAFACAEEDDGQEANDCLLIYDNEGADATGSPVGSVGCCSFIADDLDDSFLDSLGPKFKKLAEISLGVDGEGKEVQPPSKDSGYGIESCGHPIEVQQTGFVKCQTLSGSQGASALSTSGSVQPAVSIPDPLQHGNYLVTETYSASGSLVQPSTAGFDPLLTQNVIVTERVICPISSVPGNLAGPTQLRGSHTMLCTEDPCSRLI

SEQ ID NO: 38 - protein, 19 aa, Homo sapiens
EEVRKLKARVDELERIRRS

SEQ ID NO: 39 - protein, 18 aa, Homo sapiens
RIRRSILPYGDSMDRIEK

SEQ ID NO: 40 - protein, 14 aa, Homo sapiens
MDRIEKDRLQGMAP

SEQ ID NO: 41 - skipped

SEQ ID NO: 42 - protein, 24 aa, Homo sapiens
RALNSMGQDLERPLELRVRVLDIN

SEQ ID NO: 43 - protein, 25 aa, Homo sapiens
FKIIRQEPSDSPMFIINRNTGEIRT

SEQ ID NO: 44 - protein, 18 aa, Homo sapiens
NERTNVGILKVVKPLDYE

SEQ ID NO: 45 - protein, 26 aa, Homo sapiens
INVREGIAFRPASKTFTVQKGISSKK

SEQ ID NO: 46 - protein, 20 aa, Homo sapiens
REGEDNSKRNPIAKITSDYQ

SEQ ID NO: 47 - protein, 19 aa, Homo sapiens
GTSYPTTSPGTRYGRPHSG

SEQ ID NO: 48 - protein, 16 aa, Homo sapiens
KFVKNMNRDSTFIVNK